(19) United States
(12) Patent Application Publication
(10) Pub. No.: US 2016/0278740 A1
(43) Pub. Date: Sep. 29, 2016

(54) ULTRASOUND IMAGING SYSTEM AND METHOD

(71) Applicant: Hyland Software, Inc., Westlake, OH (US)

(72) Inventors: Cristian Negrila, Fairview Park, OH (US); Barbara Sardinha, Portsmouth, RI (US); Brian Sable, Euclid, OH (US); Terese A. Davis, Elyria, OH (US)

(21) Appl. No.: 14/665,194

(22) Filed: Mar. 23, 2015

(51) Int. Cl. A61B 8/00; A61B 8/08; A61B 8/14

(52) U.S. Cl. CPC A61B 8/468; A61B 8/14; A61B 8/0866; A61B 8/5223

(57) ABSTRACT

An ultrasound imaging and reporting system includes an ultrasound transducer probe, a processor component, and a data storage component. The processor component is configured to execute instructions to process ultrasound image information received from the ultrasound transducer probe into an ultrasound image and store the ultrasound image on a data storage component. The processor component is also configured to receive examination information from an input device, to store the examination information in the data storage component, and to automatically insert, at least a portion of the examination information into a main ultrasound examination report and an additional examination report stored on the data storage component. A method for operating the system is also included.

### FIELD

This disclosure relates to ultrasound technology, such as fetal ultrasound technology.

### BACKGROUND

Physicians or health care professionals who administer ultrasound examinations or interpret the ultrasound imaging are faced with a number of reports that should be completed and sent to the patient or other specialists. The completion of these reports can be time-consuming and repetitive, and this can create a conflict considering that time spent using expensive ultrasound imaging equipment must be efficiently used to primarily acquire images.

This conflict may cause difficulties for physicians and health care professionals, particularly in busy offices. Completing multiple reports while performing ultrasound exams may not be the most efficient use of time in the examination room. Because of this often the reports may be completed later when the patient has left. Even when a main ultrasound exam report is completed in real time, other reports or documentation, such as patient reports (e.g. a perinatal consult letter), or reports to a patient's specialist (e.g. an obstetrician) or primary care physician may only be completed later in time. In addition to the extra time and additional computer processing required to review images and complete reports in a serial manner, this work flow can lead to difficulties in remembering details, inconsistencies, or inaccuracies in information on the reports.

### SUMMARY

In an embodiment, an ultrasound imaging and reporting system includes an ultrasound transducer probe, a processor component, and a data storage component. The processor component is configured to execute instructions to process ultrasound image information received from the ultrasound transducer probe into an ultrasound image and store the ultrasound image on a data storage component. The processor component is also configured to receive examination information from an input device, to store the examination information in the data storage component, and to automatically insert, at least a portion of the examination information into a main ultrasound examination report and an additional examination report stored on the data storage component. This is meant to include as one embodiment, the situation where the examination information received from the input device is immediately displayed in the main examination report and then at least a portion of that examination information is automatically inserted, into the additional examination report either immediately or later when the additional examination report is displayed.

In an embodiment, a method for operating an ultrasound imaging and reporting system includes: receiving an ultrasound image from an ultrasound machine and storing the ultrasound image on a data storage component; receiving examination information from an input device component and storing the examination information on the data storage component; and automatically inserting, at least a portion of the examination information into the main examination report and an additional examination report.

An ultrasound imaging reporting system includes a processor component that is configured to execute instructions to receive an ultrasound image from an ultrasound machine and store the ultrasound image on a data storage component; the processor component is configured to receive examination information received from an input device and store the examination information in the data storage component. The processor component is configured to execute instructions to automatically insert, at least a portion of the examination information into a main examination report and an additional examination report stored on the data storage component.

The articles “a,” “an,” and “the” should be interpreted to mean “one or more” unless the context clearly indicates the contrary. The term “set” as used herein, should be interpreted to mean a set with at least one member, and not an empty set, unless the context clearly indicates the contrary. Additionally, as used herein, the term “exemplary” is intended to mean serving as an illustration or example of something, and is not intended to indicate a preference.

As used herein, the term “system” is intended to encompass computer-readable data storage that is configured with computer-executable instructions that cause certain functionality to be performed when executed by a processor. The computer-executable instructions may include a routine, a function, or the like. It is also to be understood that a system may be localized on a single device or distributed across several devices.

### BRIEF DESCRIPTION OF THE DRAWINGS

FIG. 1 is a diagram of an embodiment of an ultrasound imaging and reporting system.

FIG. 2 is an image depicting an example of the fields and reports including those fields that may be in the examination reports disclosed herein.

FIG. 3 is an image depicting an embodiment of a portion of a graphical user interface for use with the systems and methods disclosed herein.

FIG. 4 depicts a flow diagram of an embodiment of a method of operating an ultrasound imaging and reporting system.

FIG. 5 is a general diagram of an exemplary computing device on which features and steps of the presently described technology may be implemented on.

### DETAILED DESCRIPTION

The present disclosure sets forth a computer-implemented system for an ultrasound machine to process multiple examination reports in parallel, to reduce inconsistencies, errors, and processing time in a practitioner's workflow, where the multiple examination reports are created for a patient ultrasound examination.

Absent the features described herein, a physician faced with completing multiple reports per examination, would need to perform the additional required reports or documentation in a serial manner, one after another, performing repetitive work, reviewing and possibly opening the same images multiple times (a time-consuming and processor intensive task), and keying the same information multiple times, in order to duplicate certain information from report to report. Each report would also need to be finalized by signing off on them separately.

The serial processing of these examination reports takes longer and is more processor intensive than the parallel processing method described herein, impacting negatively the physician's productivity. In addition, the manual duplication of the same information from report to report can be a consistent source of discordance between these reports because the typographical errors inherent to the manual keying process.

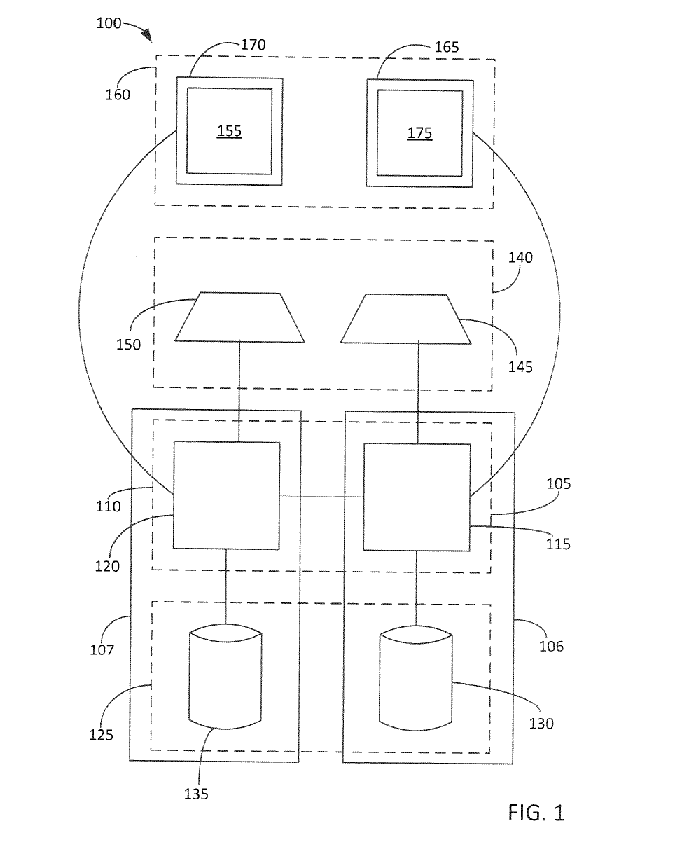

An embodiment of an ultrasound imaging and reporting system 100 is shown in FIG. 1. The system 100 includes an ultrasound transducer probe 105 that is manipulated by a health care professional (sometimes called a “sonographer”) to an area of analysis on a patient. The area of analysis is commonly a fetus, but it may also be an internal organ, or an area of an injury, such as a soft tissue injury, or other areas of a body where ultrasound images are taken for diagnostic purposes.

The system 100 can be a single ultrasound machine or an ultrasound machine with one or more additional computing devices attached, e.g., an ultrasound computing device 106 and a reporting computing device 107. To account for both situations the electronic devices are discussed in terms of components that may include one or multiple processors, data storages, and other electronic devices.

The ultrasound transducer probe 105 is coupled to a processor component 110. The processor component 110 may comprise more than one processor, such as a first processor 115 and a second processor 120. The first processor 115 may, in an embodiment, perform ultrasound machine functionality while the second processor 120 performs reporting functionality. The first processor 115 and second processor 120 may operate on the same or different physical computing devices and are in communication, such as through a cable, a network, or a wireless connection. In an embodiment, the communication is via a DICOM protocol. As is detailed in the following description, in an embodiment, the ultrasound imaging and reporting system 100 operates on an ultrasound computing device 106 and partly or fully on another (second) reporting computing device 107 connected to the ultrasound computing device 106.

In an embodiment, a program implementing the same functionality can reside on the ultrasound computing device 106 as well as on the reporting computing device 107, and the two instances of the program can share data and data storage. In another embodiment, the program for reporting resides on the reporting computing device 107 and the program for processing an ultrasound signal information received from the transducer probe 105 resides on the ultrasound computing device 106.

The system 100 also includes a data storage component 125 that is a computer-readable medium that stores instructions for execution by the processor component 110 to operate the system 100. The data storage component 125 may comprise more than one data storage, such as a first data storage 130 and a second data storage 135, which, in an embodiment, may be in communication with, respectively, the first processor 115 and the second processor 120.

In an embodiment, the processor component 110 is operable to command the ultrasound transducer probe 105 to emit ultrasound signals. The ultrasound transducer probe 105 then receives ultrasound image information and communicates it to the processor component 110. The processor component 110 is configured to execute instructions to process the ultrasound image information received from the ultrasound transducer probe 105 into an ultrasound image and store the ultrasound image on a data storage component 130. In an embodiment, the instructions to process ultrasound image information received from the ultrasound transducer probe 105 are stored in the first data storage 130 and are executed on the first processor 115, and the ultrasound image thus obtained from the processing is first stored on the first data storage 130, and is then communicated to the second processor 120 and stored on the second data storage 135.

The processor component 110 is also configured to receive examination information from an input device component 140, (for example, a keyboard, mouse, touchscreen, or other control devices), and execute instructions to store the examination information in the data storage component 125.

In an embodiment, the input device component 140 comprises a first input device 145 and a second input device 150, and the second input device 150 is in communication with the second processor 120 and the second processor 120 receives the examination information and executes instructions to store the examination information in the second data storage 135. In an embodiment, the first input device 145 is in communication with the first processor 115, and the first processor 115 is configured to receive input from the first input device 145, such as examination information, which may be stored in the first data storage 130 and later communicated to the second processor 120 and stored on the second data storage 135. In an embodiment, the input device component 140, for example, the first input device 145, may also communicate instructions for controlling parameters of the ultrasound transducer probe 105. In an embodiment, some or all of the examination information is entered on the second input device 150 and received by the second processor 120, and is stored in the second data storage 135.

The processor component 110 is configured to execute instructions to process a main ultrasound examination report and an additional examination report in a parallel manner. For example, the processor component 110 executes instructions to automatically insert, in some embodiments without user interaction, at least a portion of the examination information received from the input device component 140 into a main ultrasound examination report and an additional examination report stored on the data storage component 125. In an embodiment this happens simultaneously without user interaction. The processor component 110 may also execute instructions to automatically insert, without user interaction, the ultrasound image or a modified version thereof into at least the main ultrasound report. This may be performed by the processor component 110 by, for example, through a set of computer-executable instructions. In an embodiment, the ultrasound image may be modified by, for example, resizing, zooming, re-centering, or otherwise performing image editing on it.

At the data storage component 125 the examination information and ultrasound image may be stored in a data structure associated with fields of the main ultrasound examination report and fields of the additional examination reports that are common to the main ultrasound examination report. Alternatively, the examination information and ultrasound image may be stored in a data structure associated with fields of the main ultrasound examination report, and upon receiving a command to access the additional examination report, the processor component 110 parses the examination information in data storage and automatically fills in fields of the additional examination report with the examination information and ultrasound image, where that information is in fields that are common to the main ultrasound examination report. It should be understood as stated above, that “an ultrasound image” means one or more ultrasound images.

In an embodiment, the processor component 110 executes instructions to cause the ultrasound imaging system 100 to present a reporting graphical user interface 155 on a display device component 160 that facilitates reporting of an ultrasound exam. In an embodiment, the display device component 160 comprises a first display device 165 and a second display device 170 that each may display a different graphical user interface. For example, in an embodiment, the reporting graphical user interface 155 is displayed on the second display device 170, and an ultrasound imaging graphical user interface 175 is displayed on the first display device 165.

In an embodiment, the processor component 110 executes instructions to cause the graphical user interface to display the main examination report in one tab on a portion of the screen of a display device component 160, and one or more additional examination reports in another tab on a portion of the screen of a display device component 160. In an embodiment, when input is received to display an additional examination report that was not previously shown, the processor component 110 in response executes instructions to either (1) retrieve from the data storage and display the requested examination report with at least a portion of the examination information already entered in fields of the additional examination report; or (2) to parse the examination information in data storage associated with the main examination report, for information that is associated with the fields in the additional examination report, and retrieve and automatically display the additional examination report with that information already entered into the fields of the additional examination report. In the first option, the examination information is proactively and duplicatively stored in multiple examination reports in the data storage component 125, such as when it is initially entered in the main examination report. In the second option, the examination information is not stored in the additional examination report in the data storage component 125 until right before the additional examination report is displayed.

The processor component 110 is also configured to receive input that finalizes the main ultrasound examination report and all additional examination reports simultaneously. Upon finalization, further changes to the main examination report and all additional examination reports are prohibited without receiving additional authorizing input. The significance of this is explained in more detail below.

In an embodiment, the ultrasound imaging and reporting system 100 is designed to work with a system that allows creating customized templates for examination reports. For example, the system 100 may accept input to select fields for an ultrasound examination report and save them as a template for an ultrasound examination report. The created template can be designated as a main examination report, where, for example, the fields are selected to cover all elements of an examination that are relevant to the user's practice. These fields may differ for practice areas such as, for example, fetal ultrasound, abdominal or internal organ ultrasound, and soft tissue injuries. The fields of a main examination report should be selected to have information directly keyed in or imported from the ultrasound machine.

One or more additional examination reports may also be created that include some of the same fields as the main examination report, and, as such, will include identical data, once data is entered into the fields in the main examination report. An additional report may also include different fields that are not in the main examination report. These non-duplicate fields will require additional data entry or input from the practitioner directly in the additional report.

In an embodiment, with a multiple reports structure, a user may create a group of reports by adding additional reports (e.g., by associating as additional tabs on a graphical user interface) to an existing main report stored in the data storage component 125. A different set of additional tabs may be defined for each type of main examination report.

In accordance with this template-based reporting feature, the processor component 110 may be configured to execute instructions to accept input to create a template-based examination report, wherein the template-based examination report is stored in the data storage component 125 as a main examination report or an additional examination report. In addition, a group of main and additional reports may also be stored in the data storage component 125.

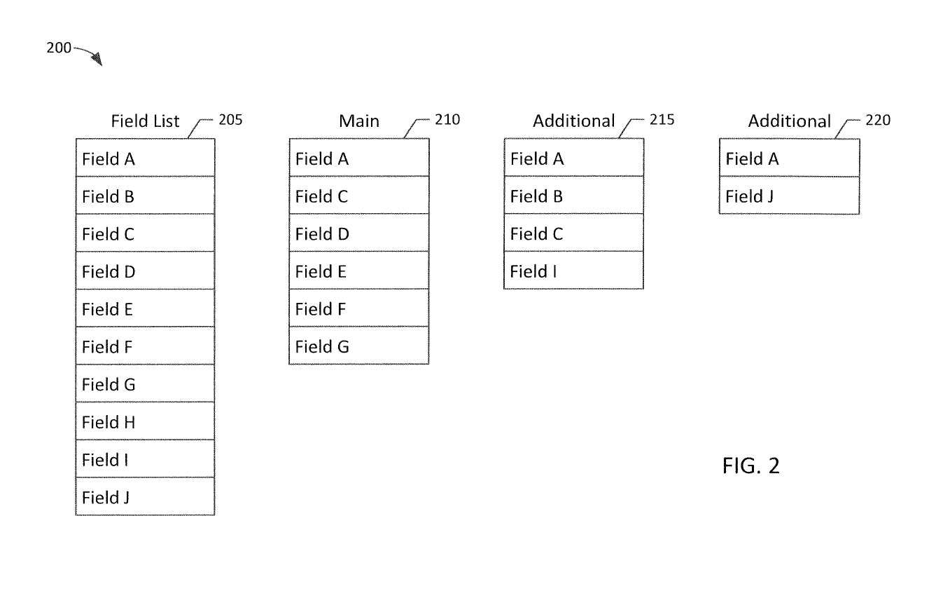

FIG. 2 shows an example of a field list 205 and how the fields may be selected to be stored in the main examination report 210, a first additional examination report 215, and a second additional examination report 220. In this example, the main examination report 210 includes a majority of the fields available from the field list 205. Certain fields that are duplicates from the main examination report are also in both the first additional examination report 215 and the second additional examination report 220. In addition, in this example, both the first additional examination report 215, and the second additional examination report 220 also include fields that are not in the main examination report. In an embodiment, a field, such as Field G, may call for the insertion of an ultrasound image. The main examination report 210, a first additional examination report 215, and a second additional examination report 220 may be stored in data storage as an examination report group.

Table 1 shows a list of examples of fields that may be selected to be included in a main examination report or an additional examination report. The fields are sub-categorized by data level and template type. The sample column indicates examples of one or more entries that may be made in the course of the examination. The custom questions field indicates fields that may be created by the user. The data level and template type of a custom question may be designated by the user also. In an embodiment, the main examination report includes the fields in Table 1, and an additional examination report includes a subset of the fields in the main examination report.

TABLE 1

| | Data | Template | |
|---|---|---|---|
| Field | Level | Type | Sample |
| Custom | Any | Any | |
| Questions | | | |
| Placeholder | Patient | All | Manually entered text |
| Patient Name | Patient | All | Jane M. Doe |
| MRN | Patient | All | 123456789 |
| (Medical | | | |
| Record | | | |
| Number) | | | |
| MPI | Patient | All | 987654321 |
| (Master Patient | | | |
| Index) | | | |
| DOB | Patient | All | Jan. 1, 1975 |
| Patient Address | Patient | All | 123 Any Street |
| | | | Any Town, OH 12345 |
| Patient Race | Patient | All | Caucasian |
| Total Visits | Patient | All | 2 |
| Current | Patient | All | Dr. Mary Referring1, M.D. |
| Referring | | | |
| Physician 1 | | | |
| Current | Patient | All | Dr. Mary Referring2, M.D. |
| Referring | | | |
| Physician 2 | | | |
| Current | Patient | All | Dr. Mary Referring3, M.D. |
| Referring | | | |
| Physician 3 | | | |
| Current | Patient | All | 123 Any Street |
| Referring | | | Suite B |
| Address 1 | | | Any Town, OH 12345 |
| Current | Patient | All | 123 Any Street |
| Referring | | | Suite B |
| Address 2 | | | Any Town, OH 12345 |
| Current | Patient | All | 123 Any Street |
| Referring | | | Suite B |
| Address 3 | | | Any Town, OH 12345 |
| Number Of | Exam/Consult | OB Exam | 2 |
| Fetuses | | | |
| Fetus Pregnancy | Exam/Consult | OB Exam | SINGLE/TWIN/TRIPLET/QUADRUPLET/ |
| | | | QUINTUPLET/SEXTUPLET/SEPTUPLET/ |
| | | | OCTUPLET |
| Assigned EGA | Exam/Consult | OB Exam | 21 6/7 |
| (Estimated | | | |
| Gestational | | | |
| Age) | | | |
| Assigned EGA | Exam/Consult | OB Exam | LMP |
| Calculated By | | | |
| LMP | Exam/Consult | OB Exam | Jan. 1, 2009 |
| | | | Uncertain |
| | | | Not Available |
| LMP Macro | Exam/Consult | OB Exam | Her LMP was Jan. 1, 2009. |
| | | | Her LMP was uncertain. |
| | | | Her LMP was not available. |
| Gravidity | Exam/Consult | OB Exam | 35 y/o G1234P5 E1 |
| EDD | Exam/Consult | OB Exam | Oct. 1, 2009 |
| (Expected Date | | | |
| of Delivery) | | | |
| Conception | Exam/Consult | OB Exam | Jan. 1, 2009 |
| Date | | | |
| Discordance | Exam/Consult | 2nd/3rd | 8% |
| | | Trimester | |
| | | & Custom | |
| | | OB | |
| Exam Type | Exam/Consult | Any | Detailed Survey |
| Consulting | Exam/Consult | Consult | Dr. Bob Signature, M.D. |
| Physician | | | |
| Consulting | Exam/Consult | Consult | Director of Perinatal Research |
| Physician Title | | | |
| Consult Title | Exam/Consult | Consult | Consult Report Title |
| Consult Tag | Exam/Consult | Consult | Consult Tag |
| Consult Date | Exam/Consult | Consult | Jan. 1, 2009 |
| Exam Date | Exam/Consult | Any | Jan. 1, 2009 |
| | | Exam | |
| Visit Number | Exam/Consult | Any | 2 |
| | | Exam | |
| Indications | Exam/Consult | Any | ectopic pregnancy suspected |
| | | Exam | |
| Accession | Exam/Consult | Any | AB123456 |
| Number | | Exam | |
| Exam Location | Exam/Consult | Any | Main Location |
| | | Exam | |
| Patient Age | Exam/Consult | Any | 25 years old |
| | | Exam | |
| Interpreting | Exam/Consult | Any | Dr. John Interpreting1, M.D. |
| Physician 1 | | Exam | |
| Interpreting | Exam/Consult | Any | Dr. John Interpreting2, M.D. |
| Physician 2 | | Exam | |
| Exam Referring | Exam/Consult | Any | Dr. Mary Referring1, M.D. |
| Physician 1 | | Exam | |
| Exam Referring | Exam/Consult | Any | Dr. Mary Referring2, M.D. |
| Physician 2 | | Exam | |
| Exam Referring | Exam/Consult | Any | Dr. Mary Referring3, M.D. |
| Physician 3 | | Exam | |
| Gyn LMP | Exam/Consult | Gynecology | Jan. 1, 2009 |
| | | | uncertain |
| Gyn LMP | Exam/Consult | Gynecology | Her LMP was Jan. 1, 2009. |
| Macro | | | Her LMP was uncertain. |
| | | | She is post-menopausal. |
| Gyn Gravidity | Exam/Consult | Gynecology | 35 y/o G1234P5 E1 |
| Gyn Ovary-Left | Exam/Consult | Gynecology | Appears normal/Suboptimal visualization/Appears |
| | | | abnormal/Removed/Not Seen |
| Gyn Ovary-Left | Exam/Consult | Gynecology | 123.45 × 234.56 × 345.67 mm |
| Measurement | | | |
| Gyn Ovary- | Exam/Consult | Gynecology | Appears normal/Suboptimal visualization/Appears |
| Right | | | abnormal/Removed/Not Seen |
| Gyn Ovary- | Exam/Consult | Gynecology | 123.45 × 234.56 × 345.67 mm |
| Right | | | |
| Measurement | | | |
| Uterus | Exam/Consult | Gynecology | Non gravid |
| | | | Pregnant |
| | | | Removed in 2007 |
| | | | Post-menopausal since 2008 |
| Gyn Uterus | Exam/Consult | Gynecology | 123.45 × 234.56 × 345.67 mm |
| Measurement | | | |
| Uterus Volume | Exam/Consult | Gynecology | 1234 cc |
| Uterus Position | Exam/Consult | Gynecology | Anteverted/Anteflexed/Retroverted/Retroflexed |
| Gyn Uterus | Exam/Consult | Gynecology | normal |
| Appearance | | | abnormal due to Didelphys |
| Endometrium | Exam/Consult | Gynecology | 123.45 mm |
| Double | | | |
| Thickness | | | |
| Endometrium | Exam/Consult | Gynecology | Trilaminar, Hyperechoic, Ill-defined |
| Texture | | | |
| Endometrial | Exam/Consult | Gynecology | normal |
| Cavity | | | abnormal due to global endometrial hyperplasia |
| | | | abnormal due to a blood clot measuring |
| | | | 123 × 234 × 345 mm |
| Cervix | Exam/Consult | Gynecology | visualized/sub optimally visualized |
| Visualized | | | |
| Cervix | Exam/Consult | Gynecology | normal/abnormal |
| Appearance | | | |
| Cervix Findings | Exam/Consult | Gynecology | Nabothian Cyst/Other |
| Ovary-Left | Exam/Consult | 1st or 2nd | Normal/Suboptimal/Abnormal/Removed/Not Seen |
| | | TM Exam | |
| Ovary-Left | Exam/Consult | 1st or 2nd | 123.45 × 234.56 × 345.67 mm |
| Measurement | | TM Exam | |
| Ovary-Right | Exam/Consult | 1st or 2nd | Normal/Suboptimal/Abnormal/Removed/Not Seen |
| | | TM Exam | |
| Ovary-Right | Exam/Consult | 1st or 2nd | 123.45 × 234.56 × 345.67 mm |
| Measurement | | TM Exam | |
| Cervix Seen | Exam/Consult | 1st or 2nd | visualized/sub optimally visualized |
| | | TM Exam | |
| Cervical | Exam/Consult | 1st or 2nd | normal/shortened, dilated, funneled and hourglass |
| Appearance | | TM Exam | |
| Cervix Macro | Exam/Consult | 1st or 2nd | The cervix was visualized, appearing dilated, and |
| | | TM Exam | measures 123 mm. |
| | | | There was suboptimal visualization of the cervix. |
| Uterus | Exam/Consult | 1st or 2nd | normal |
| Appearance | | TM Exam | abnormal due to suspected uterine didelphys |
| Uterus | Exam/Consult | 1st or 2nd | L-123.45 mm W-234.56 mm H-345.67 mm |
| Measurement | | TM Exam | |
| Adnexal Mass | Exam/Consult | 1st or 2nd | An adnexal mass was noted. |
| Noted | | TM Exam | An adnexal mass was not noted. |
| Cul De Sac | Exam/Consult | 1st or 2nd | Fluid was noted in the Cul-de-sac measuring |
| | | TM Exam | 123.45 mm × 234.56 mm × 345.67 mm. |
| | | | There was no free fluid noted in the Cul-de-sac. |
| Cervical Overall | Exam/Consult | 2nd/3rd | 123 mm |
| Length | | Trimester | |
| Dilation of | Exam/Consult | 2nd/3rd | 123 mm |
| internal os | | Trimester | |
| Funnel Length | Exam/Consult | 2nd/3rd | 123 mm |
| | | Trimester | |
| Funnel Width | Exam/Consult | 2nd/3rd | 123 mm |
| | | Trimester | |
| Functional | Exam/Consult | 2nd/3rd | 123 mm |
| Length | | Trimester | |
| Cervical Index | Exam/Consult | 2nd/3rd | 123 mm |
| | | Trimester | |
| Cervical Length | Exam/Consult | 1st | 123 mm |
| | | Trimester | |
| Internal OS | Exam/Consult | 1st | 123 mm |
| | | Trimester | |
| 1st TM Funnel | Exam/Consult | 1st | 123 mm |
| Length | | Trimester | |
| Endocervical | Exam/Consult | 1st | 123 mm |
| Canal Length | | Trimester | |
| 1st TM Cervical | Exam/Consult | 1st | 123 mm |
| Index | | Trimester | |
| 1st TM EGA by | Exam/Consult | 1st | 9 6/7 |
| US | | Trimester | |
| Estimated Fetal | Fetus | 2nd/3rd | 123 grams |
| Weight-Single | | Trimester | |
| Fetus | | & Custom | |
| | | OB | |
| EFW | Fetus | 2nd/3rd | 48 |
| (Estimated Fetal | | Trimester | |
| Weight | | & Custom | |
| Percentile)- | | OB | |
| Single Fetus | | | |
| Presentation- | Fetus | 2nd/3rd | Cephalic |
| Single Fetus | | Trimester | Incomplete Breech |
| | | | Transverse, spine up, head right |
| Presentation-All | Fetus | 2nd/3rd | Fetus A is in the Cephalic position. Fetus B is in the |
| Fetuses | | Trimester | Complete Breech position. |
| Placenta | Fetus | 2nd/3rd | Anterior, left, fundal |
| Position-Single | | Trimester | Posterior, right |
| Fetus | | | |
| Placenta | Fetus | 2nd/3rd | The placenta is Anterior, Left, Fundal, grade 1. |
| Position Macro- | | Trimester | There is marginal placenta previa. |
| Single Fetus | | | The placenta is Posterior, Right. There is no |
| | | | evidence of placenta previa. |
| Placenta | Fetus | 2nd/3rd | The placenta for Fetus A is Anterior, Left, Fundal, |
| Position Macro- | | Trimester | grade 1. There is marginal placenta previa. |
| All Fetuses | | | The placenta for Fetus A is Posterior, Right. There |
| | | | is no evidence of placenta previa. |
| Placentation- | Fetus | 2nd/3rd | Normal |
| Single Fetus | | Trimester | Suspicious for accreta, increta, percreta |
| Placenta | Fetus | 2nd/3rd | The placenta is abnormal in appearance due to |
| Abnormal | | Trimester | placental lake. |
| Macro-Single | | | The placenta is abnormal in appearance due to |
| Fetus | | | Retromembranous Collection and Succenturiate |
| | | | Lobe. |
| Placenta | Fetus | 2nd/3rd | The placenta for Fetus A is abnormal in appearance |
| Abnormal | | Trimester | due to placental lake. |
| Macro-All | | | The placenta for Fetus A is abnormal in appearance |
| Fetuses | | | due to Retromembranous Collection and |
| | | | Succenturiate Lobe. |
| Subjective AF | Fetus | 2nd/3rd | Normal |
| (Amniotic | | Trimester | Mildly Decreased |
| Fluid) | | | Moderately Decreased |
| Volume-Single | | | Markedly Decreased |
| Fetus | | | Mildly Increased |
| | | | Moderately Increased |
| | | | Markedly Increased |
| Overall | Fetus | 2nd/3rd | Normal/Anhydramnios/Oligohydramnios/ |
| Assessment- | | Trimester | Polyhydramnios |
| Single Fetus | | | |
| Maximum | Fetus | 2nd/3rd | 12 cm/mm |
| Vertical Pocket- | | Trimester | |
| Single Fetus | | | |
| AFI-Single | Fetus | 2nd/3rd | 123 cm/mm |
| Fetus | | Trimester | |
| Placental Cord | Fetus | 2nd/3rd | normal/marginal/velamentous/suboptimal |
| Insertion-Single | | Trimester | |
| Fetus | | | |
| Umbilical Cord | Fetus | 2nd/3rd | The fetus has a 3 vessel cord. |
| Macro-Single | | Trimester | There was suboptimal visualization of the umbilical |
| Fetus | | | cord. |
| | | | The umbilical cord for the fetus is abnormal (2 VC, |
| | | | shortened). |
| Umbilical Cord | Fetus | 2nd/3rd | Fetus A has a 3 vessel cord. |
| Macro-All | | Trimester | There was suboptimal visualization of the umbilical |
| Fetuses | | | cord for Fetus B. |
| | | | The umbilical cord for fetus C is abnormal (2 VC, |
| | | | shortened). |
| Cervical | Fetus | 2nd/3rd | A transabdominal cervical evaluation was |
| Evaluation | | Trimester | performed. |
| Macro | | | A cervical evaluation was not performed. |
| Dynamic | Fetus | 2nd/3rd | There were spontaneous dynamic changes. |
| Changes Macro | | Trimester | There were dynamic changes after fundal pressure. |
| | | | There were no dynamic changes. |
| Suboptimal | Fetus | 2nd/3rd | Calvarium, Cerebellum and Midline Falx |
| Anatomy-Single | | Trimester | |
| Fetus | | | |
| Not Visualized | Fetus | 2nd/3rd | Profile and Orbits |
| Anatomy-Single | | Trimester | |
| Fetus | | | |
| Abnormal | Fetus | 2nd/3rd | Cervical Spine (SCOLIOSIS/KYPHOSIS) and Left |
| Anatomy-Single | | Trimester | Toes (SYNDACTYLY, POLYDACTYLY) |
| Fetus | | | |
| Gestational Sac | Fetus | 1st | was seen/was not seen |
| Seen-Single | | Trimester | |
| Fetus | | | |
| Gestational Sac | Fetus | 1st | a normal position within the uterus |
| Location-Single | | Trimester | an abnormal position within the uterus |
| Fetus | | | an extrauterine location |
| Gestational Sac | Fetus | 1st | 12 × 34 × 56 mm |
| Measurements- | | Trimester | |
| Single Fetus | | | |
| Gestational Sac | Fetus | 1st | 34 mm (10 2/7 wks) |
| Mean-Single | | Trimester | |
| Fetus | | | |
| Gestational Sac | Fetus | 1st | A gestational sac was seen and measures 34 mm (10 |
| Macro-Single | | Trimester | 2/7 wks). The gestational sac has an abnormal |
| Fetus | | | position within the uterus. |
| Gestational Sac | Fetus | 1st | A gestational sac was seen for fetus A and measures |
| Macro-All | | Trimester | 34 mm (10 2/7 wks). The gestational sac for Fetus A |
| Fetuses | | | has an abnormal position within the uterus. |
| Yolk Sac | Fetus | 1st | 12.34 mm |
| Measurement- | | Trimester | |
| Single Fetus | | | |
| Yolk Sac | Fetus | 1st | A yolk sac was seen and measures 12.34 mm. |
| Macro-Single | | Trimester | A yolk sac was not seen. |
| Fetus | | | |
| Yolk Sac | Fetus | 1st | A yolk sac was seen for fetus A and measures 12.34 |
| Macro-All | | Trimester | mm. |
| Fetuses | | | A yolk sac for fetus A was not seen. |
| CRL | Fetus | 1st | 123.4 mm (9 4/7 wks) |
| Measurement- | | Trimester | |
| Single Fetus | | | |
| Fetal Pole Seen- | Fetus | 1st | visualized/sub optimally visualized/not visualized |
| Single Fetus | | Trimester | |
| Fetal Pole | Fetus | 1st | A 123.4 mm fetal pole was visualized and is equal |
| Macro-Single | | Trimester | to 9 weeks and 4 days gestation. |
| Fetus | | | A fetal pole was not visualized. |
| | | | There was suboptimal visualization of the fetal pole. |
| Fetal Pole | Fetus | 1st | A 123.4 mm fetal pole was visualized for fetus A |
| Macro-All | | Trimester | and is equal to 9 weeks and 4 days gestation. |
| Fetuses | | | A fetal pole was not visualized for fetus A. |
| | | | There was suboptimal visualization of the fetal pole |
| | | | for fetus A. |
| Fetal Heart | Fetus | 1st | 123 bpm |
| Rate-Single | | Trimester | |
| Fetus | | | |
| Fetal Heart | Fetus | 1st | present |
| Activity-Single | | Trimester | absent |
| Fetus | | | |
| Fetal Heart | Fetus | 1st | Fetal heart activity was present with a fetal heart rate |
| Activity Macro- | | Trimester | of 98 bpm. |
| Single Fetus | | | Fetal heart activity was absent. |
| Fetal Heart | Fetus | 1st | Fetal heart activity was present for fetus A with a |
| Activity Macro- | | Trimester | fetal heart rate of 98 bpm. |
| All Fetuses | | | Fetal heart activity was absent for fetus A. |
| Nuchal | Fetus | 1st | 12.34 mm |
| Translucency | | Trimester | |
| Measurement- | | | |
| Single Fetus | | | |
| Nuchal | Fetus | 1st | visualized/ |
| Translucency | | Trimester | suboptimal visualization |
| Seen-Single | | | |
| Fetus | | | |
| Nuchal | Fetus | 1st | The nuchal translucency was visualized and |
| Translucency | | Trimester | measures 12.34 mm. |
| Macro-Single | | | There was suboptimal visualization of the nuchal |
| Fetus | | | translucency. |
| Nuchal | Fetus | 1st | The nuchal translucency was visualized for fetus A |
| Translucency | | Trimester | and measures 12.34 mm. |
| Macro-All | | | There was suboptimal visualization of the nuchal |
| Fetuses | | | translucency for fetus A. |
| Fluid | Fetus | 1st | 123 × 234 × 345 mm |
| Collections- | | Trimester | |
| Single Fetus | | | |
| Sac Location- | Fetus | 1st | Right, Superior, Anterior |
| Single Fetus | | Trimester | Left, Inferior, Posterior |
| Placenta | Fetus | 1st | Anterior, Left, Fundal, Diffuse |
| Position (1st | | Trimester | Posterior, Right |
| TM)-Single | | | |
| Fetus | | | |
| Placenta Macro | Fetus | 1st | Fetus A: Anterior, left with placentamegaly. Fetus |
| | | Trimester | B: Posterior, right. |
| | | | Posterior, right with Retromembranous Collection. |
| | | | Dividing membrane was seen. Placentation is |
| | | | monochorionic, monoamniotic. |

In an embodiment, the entries in the fields of the main examination report comprise the examination information. In an embodiment, the examination information may comprise: patient demographic information, patient measurement information obtained by measuring the ultrasound image, and practitioner diagnostic notes and other miscellaneous examination information. Data filling each field of the examination information may be entered through an input device attached directly to an ultrasound computing device, or through an input device attached to a reporting computing device. When some or all of the examination information is entered and stored in the ultrasound computing device, and a separate reporting computing device is used to process and/or finalize the reports, this examination information may be communicated to the reporting computing device and automatically, without user interaction, entered into the relevant fields of the examination report.

The patient demographic information may include such things as the age, height, weight, race and medical history information of the patient. For example, this category may include fields designated in the data level “patient” in Table 1. The patient measurement information obtained by measuring the ultrasound image may include various measurements such as, for example, those in the fields of Table 1 designated under the data level “exam/consult” that have measurements in the sample column. The practitioner diagnostic notes may, for example, include information that include the impressions of the practitioner or diagnosis of a condition noticed in the examination. For example, this category may include fields designated under the data level “exam/consult” that include impressions or diagnosis rather than measurement data only. Custom fields can also be created that would fit into the above-mentioned categories. An example custom field includes a general physician's comments field. In an embodiment, the comments field forms a portion of the main examination report.

In an embodiment, all the information that is duplicated or shared from the main examination report to an additional examination report is information obtained during a current performed ultrasound examination.

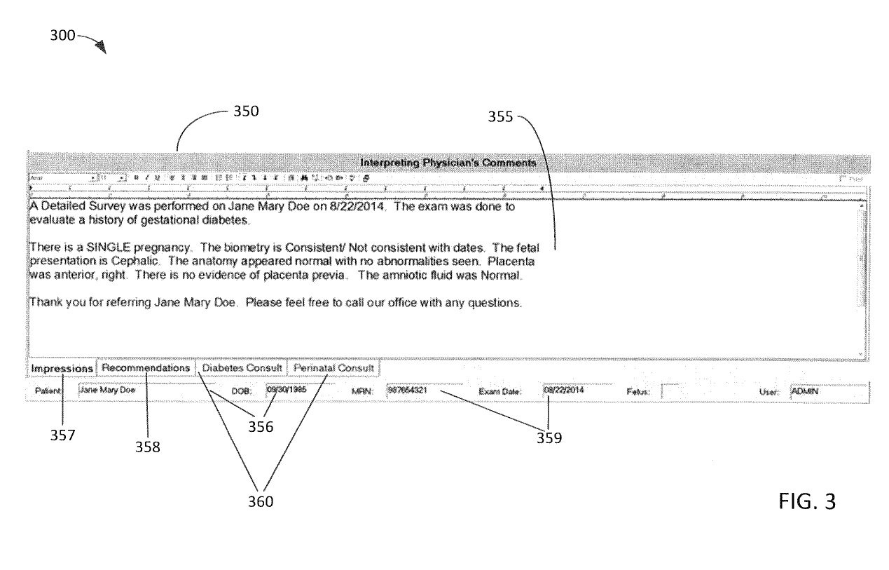

FIG. 3 shows a portion of an example graphical user interface 350. In an example, the main examination report is displayed as two tabs 357, 358 labeled “Impressions” and “Recommendations” as shown in FIG. 3. Different sets of examination information may be entered in these screens in the graphical user interface 350. In another embodiment, the main examination report is shown in a single tab on the graphical user interface 350, and includes all the examination information fields that are set for the main examination report template. Patient demographic information 356 and miscellaneous examination information 359 is shown along the bottom of the graphical user interface 350. The two tabs 360 just above the demographic information and to the right show titles of additional examination reports and these tabs can be selected to display the additional examination reports, including the fields of the examination report and the information that was already entered or copied automatically into them.

In a fetal ultrasound imaging and reporting system, a subset of the patient demographic information and patient measurement information obtained by measuring the ultrasound image includes fetal examination information and fetal measurement information. For example, the fetal examination information can include observations on the fetus or the due date of the fetus, such as, for example, the fields designated under the data level “exam/consult” that include impressions or diagnosis rather than measurement data only. The fetal measurement information can include the various measurements such as, for example, those in the fields of Table 1 designated under the data level “fetus” that have measurements in the sample column.

One or more ultrasound images may also be stored as part of the main examination report. In another embodiment, the one or more ultrasound images are also stored as part of the additional examination report.

The additional reports can be customized according to templates created and stored by the practitioner for a variety of purposes. These additional reports contain a subset of fields from the main examination report and may also include additional fields that are not part of the main examination report. In an embodiment, additional examination reports are selected from one or more of a group consisting of: a perinatal consult report, a diabetic patient log report, a telephone call memo, and a medication list. Other customized additional reports can be tailored by the physician to match their practice needs. One further example, includes a report that includes examination data and/or one or more ultrasound images relevant for reporting to a patient's general practitioner or specialist.

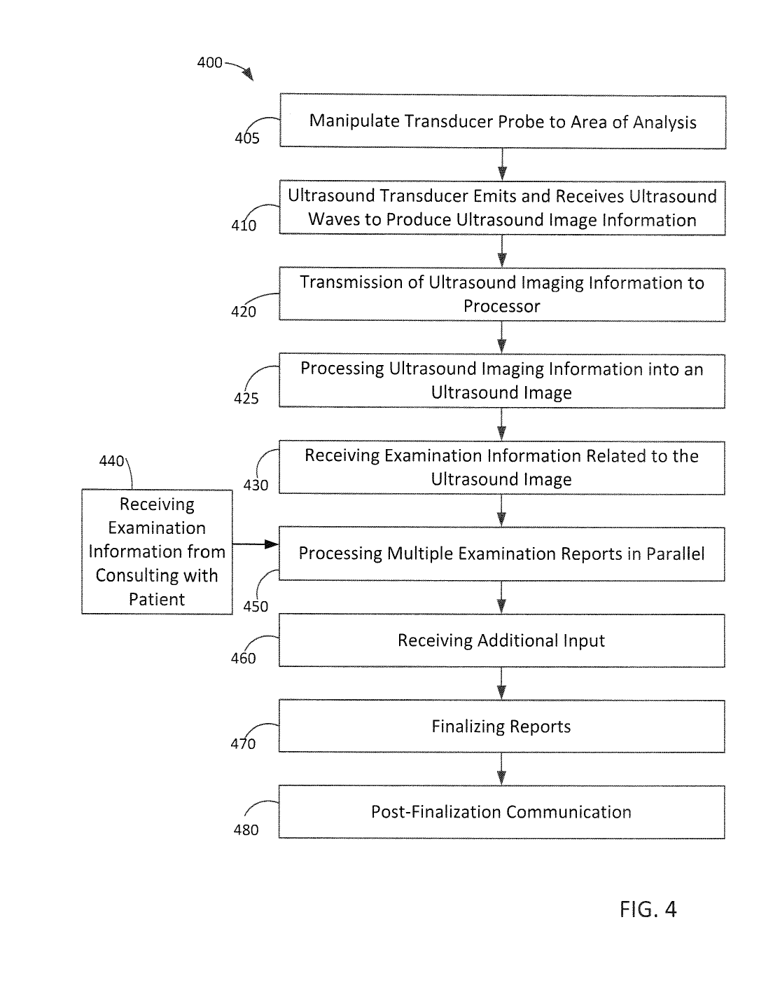

In FIG. 4 example methodologies relating to an ultrasound imaging and reporting system are shown and are described below. While the methodologies are described as being a series of acts that are performed in a sequence, it is to be understood that the methodologies are not limited by the order of the sequence, unless noted. For instance, some acts may occur in a different order than what is described herein. In addition, an act may occur concurrently with another act. Furthermore, in some instances, not all acts may be required to implement a methodology described herein.

Moreover, the acts or steps described herein may be computer-executable instructions that can be implemented by a processor component, (e.g. one or more processors) and/or stored on a data storage, (e.g. one or more computer-readable medium or media). The computer-executable instructions may include a routine, a sub-routine, programs, a thread of execution, and/or the like. Still further, results of acts of the methodologies may be stored in a data storage component, that is a computer-readable medium, displayed on a display device component, (e.g. one or more displays), through a graphical user interface component. The data storage component may be any suitable computer-readable storage device, such as memory, hard drive, CD, DVD, or flash drive. As used herein, the term “computer-readable medium” and data storage component is not intended to encompass a propagated signal. In an embodiment, the method is implemented on a single computing device or multiple computing devices, such as, for example, an ultrasound computing device and a reporting computing device. Additionally, it should be understood that a step or steps may be subdivided into sub-steps, combined together in combined steps, or combinations thereof.

In FIG. 4, an embodiment of a method of operating an ultrasound imaging and reporting system is shown. At 405 the practitioner or health care professional manipulates the ultrasound transducer probe to the area of analysis, such as, for example, a patient's uterus or a fetus. At 410 the practitioner initiates the examination by inputting a command received by a processor in communication with the probe, and the processor commands the probe to initiate emission and reception, wherein the probe emits ultrasound emissions and receives the “echo” or reflected wave to produce raw data designated herein as ultrasound imaging information. In an embodiment, the receiving and transmitting transducers are housed in the same unit, but these can also be separate units.

At 420 the ultrasound imaging information is transmitted to the processor, where, at 425, the ultrasound imaging information is processed into ultrasound images by executing instructions and algorithms known in the art. The ultrasound images are stored on a data storage component.

At 430 examination information is received by the processor. This examination information is as defined above, and may include, for example, measurements that are extracted from the ultrasound images by a practitioner reviewing and inputting information into an input device by hand or in some cases by the processor executing instructions to automatically make measurements and retrieve data from the ultrasound image or areas of the ultrasound image. Diagnostic examination information based on the practitioner's impressions of the ultrasound image may also be received from an input device.

At 440, additional examination information, such as demographic or examination information, may be gathered from consulting with the patient and inputting the examination information into an input device. In an embodiment, at least some of this examination information may also come from preexisting medical records already in data storage. Step 440 may take place at any time prior to finalization of the reports, and may even be intermittently performed as the ultrasound examination proceeds. In addition, steps 430 and 440, may partly or completely be performed on a separate processor and separate computing device (e.g. a reporting computing device) from the ultrasound computing device, as is disclosed above. In this case, the ultrasound image and some or all of the examination information may be communicated to the reporting computing device.

At 450, multiple examination reports, such as a main ultrasound examination report and an additional examination report are processed in parallel. By this it is meant that the processor component executes instructions to automatically insert, without user interaction, at least a portion of the examination information into the main ultrasound examination report and an additional examination report. The main and additional examination reports may be pre-selected and formed into a pre-formed group of reports prior to beginning the exam, or may be selected and grouped together later, for example, after step 425. In an embodiment, as examination information is entered into the main examination report via an input device, the information is displayed on a graphical user interface in fields of the main ultrasound examination report and is propagated to common fields of the additional examination reports. The examination information may also be loaded into the main examination as it is received from the ultrasound machine, in a dual computing machine embodiment.

In an embodiment, the parallel processing step 450 is performed in the background, and is not displayed on the graphical user interface until the processor receives a request to display the additional examination report on the graphical user interface and in response retrieves from the data storage component and displays the additional examination report with at least a portion of the examination information (that was input into the) already entered into fields of the additional examination report. In an alternative embodiment, upon receiving the request to display the additional examination report, the processor component parses the examination information in data storage associated with the main examination report, for shared information that is associated with the fields in the additional examination report and automatically displays the additional examination report with the shared information already entered into the fields of the additional examination report.

In an embodiment, the ultrasound image is also inserted into the main examination report, and may also be propagated to an additional examination report as well.

At 460 additional examination information may be entered that is not processed in parallel. For example, there may be additional fields in the additional reports that are unique to that report and are not shared with any other report, or not shared with the main examination report.

At 470 the reports are finalized. In particular, input is received at a processor to finalize the main ultrasound examination report, and in response the processor executes instructions to finalize the main ultrasound examination report and all additional examination reports simultaneously. A practitioner, typically a physician, will finalize the reports, after ensuring that all the information required has been provided in the reports. In an embodiment, the system may provide a warning that certain required fields are not filled out upon an attempted finalization.

In an embodiment, finalizing the reports has the consequence of locking the reports in the storage component, (i.e. locking the fields and the information in the fields), and prohibiting further changes to the main examination report and all additional examination reports without receiving additional authorizing input. In an embodiment, the reports may be viewed, printed, e-mailed, faxed, but not altered once they are finalized. The input causing the finalization may be a digital signature, and may cause an image of a doctor's signature to be included on the reports. The finalization input, may, in an embodiment, require entry of a password. The additional authorizing input may, for example, be input from an administrator or that doctor that signed the reports, and may be authenticated with a password.

In an embodiment, the additional examination reports will be considered full rights examination reports only when finalized. In an embodiment, as long as the main ultrasound report is not finalized, the access to the additional examination reports is possible only through the main ultrasound report that represents the group. After the main examination report is finalized, and accordingly the additional examination reports are finalized, each additional examination report becomes an individual entity and can be viewed, unfinalized, amended, and re-finalized independently.

In an embodiment, after being signed, the additional examination reports will be stored in the data storage component as independent entities in the system. That is, the reports are no longer grouped together and if changes are made in one report they will not be reflected in common fields in the other reports.

In an embodiment, all the additional examination reports that have user entered data will be finalized when the main examination report is finalized, and will be faxed to the referring physician when the main examination report is faxed to the referring physician, will be uploaded via, e.g., HL7 (health level 7) messages, to an electronic medical records (EMR) system when the main examination report is uploaded, and will be printed when the main ultrasound report is printed. In an embodiment, these functions can be performed for the examination reports automatically and without further user interaction upon receiving the finalization input. If changes are made to a main or additional examination report after finalization, the changed examination report may be individually re-uploaded to the EMR system.

At 480, post-finalization communication acts are performed. These may be automatically performed without user interaction after the finalization step, or may require input to initiate one or all of the communication acts. These post-finalization communication acts include for example, executing instructions to communicate billing information to responsible parties, such as, for example, insurance and patient. While billing information is typically entered prior to finalization, billing codes specific to the additional reports may also be manually modified after finalization, while maintaining the main examination report. In addition, the post-finalization communication acts may also comprise the actions of printing, faxing, and electronic messaging of the examination reports to the parties of interest, such as patient, referring doctor, specialist doctor, hospital, billing system or other medical facility.

The system is also operable to execute instructions to turn off the parallel processing capability for a specific patient examination for one or more additional examination reports. The system may also present an option to dynamically hide any of the additional examination reports of the group or to alter each of the reports default printing, faxing, and/or electronic messaging setting, so when the finalization step is performed, or any of the post-finalization printing or communication actions are initiated, only the desired examination reports of the group are printed or communicated.

It should be appreciated from the above explanation that significant time and effort is saved by the automatic processing in parallel of the multiple examination reports. Copying and pasting, switching back and forth between the views of the multiple examination reports, and duplicative data entry is eliminated or minimized. Inconsistencies in the data entry and errors are also reduced, and as a result the system and method provides better patient care and reduces the practitioner's risk of liability.

The term, “in communication,” may refer to electronic, wireless, optical or other communications through, for example, an electrical cable, a fiber optic cable, or wireless interface. Communication may be through a network, such as LAN, WAN, the Internet, a virtual network, or other communications between computing devices or components of a computing device.

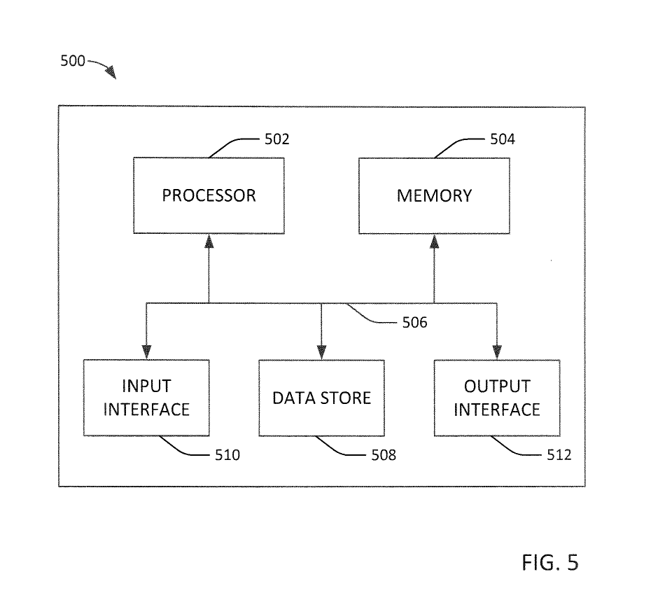

Referring now to FIG. 5, a more detailed illustration of an exemplary computing device 500 is provided that can be used in accordance with the systems and methodologies disclosed herein. For example, the exemplary computing device 500 may be the reporting computing device 107 or the ultrasound computing device 106 and encompass the processor component 110 and the data storage component 125, described in FIG. 1. However, the ultrasound transducer is in communication with the processor component.

The computing device 500 includes data storage 508 that is accessible by the processor 502 by way of the system bus 506. The data storage 508 may include executable instructions to operate the processor 502 and other components. The computing device 500 also includes an input interface 510 that allows external devices to communicate with the computing device 500. For instance, the input interface 510 may be used to receive instructions from an external computer device, from a user, etc. The computing device 500 also includes an output interface 512 that interfaces the computing device 500 with one or more external devices. For example, the computing device 500 may display text, images, etc. by way of the output interface 512.

It is contemplated that the external devices that communicate with the computing device 500 via the input interface 510 and the output interface 512 can be included in an environment that provides substantially any type of user interface with which a user can interact. Examples of user interface types include graphical user interfaces, natural user interfaces, and so forth. For instance, a graphical user interface may accept input from a user employing input device(s) such as a keyboard, mouse, remote control, or the like and provide output on an output device such as a display. Further, a natural user interface may enable a user to interact with the computing device 500 in a manner free from constraints imposed by input device such as keyboards, mice, remote controls, and the like. Rather, a natural user interface can rely on speech recognition, touch and stylus recognition, gesture recognition both on screen and adjacent to the screen, air gestures, head and eye tracking, voice and speech, vision, touch, gestures, machine intelligence, and so forth.

Additionally, while illustrated as a single system, it is to be understood that the computing device 500 may be a distributed system. Thus, for instance, several devices may be in communication by way of a network connection and may collectively perform tasks described as being performed by the computing device 500.

Various functions described herein can be implemented in hardware, software, or any combination thereof. If implemented in software, the functions can be stored on or transmitted over as one or more instructions or code on a computer-readable medium. Computer-readable media includes computer-readable storage media. A computer-readable storage media can be any available storage media that can be accessed by a computer. By way of example, and not limitation, such computer-readable storage media can comprise RAM, ROM, EEPROM, CD-ROM or other optical disk storage, magnetic disk storage or other magnetic storage devices, or any other medium that can be used to carry or store desired program code in the form of instructions or data structures and that can be accessed by a computer. Disk and disc, as used herein, include compact disc (CD), laser disc, optical disc, digital versatile disc (DVD), floppy disk, and BLU-RAY (BD), where disks usually reproduce data magnetically and discs usually reproduce data optically with lasers. Further, in an example, a propagated signal is not included within the scope of computer-readable storage media or display data. Computer-readable media also includes communication media including any medium that facilitates transfer of a computer program from one place to another. A connection, for instance, can be a communication medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio and microwave are included in the definition of communication medium. Combinations of the above should also be included within the scope of computer-readable media.

Alternatively, or in addition, the functionality described herein can be performed, at least in part, by one or more hardware logic components. For example, and without limitation, illustrative types of hardware logic components that can be used include Field-programmable Gate Arrays (FPGAs), Program-specific Integrated Circuits (ASICs), Program-specific Standard Products (ASSPs), System-on-a-chip systems (SOCs), Complex Programmable Logic Devices (CPLDs), etc.

What has been described above includes examples of one or more embodiments. It is, of course, not possible to describe every conceivable modification and alteration of the above devices or methodologies for purposes of describing the aforementioned aspects, but one of ordinary skill in the art can recognize that many further modifications and permutations of various aspects are possible. Accordingly, the described aspects are intended to embrace all such alterations, modifications, and variations that fall within the spirit and scope of the appended claims. Furthermore, to the extent that the term “includes” is used in either the details description or the claims, such term is intended to be inclusive in a manner similar to the term “comprising” as “comprising” is interpreted when employed as a transitional word in a claim.

It is claimed:

1. An ultrasound imaging and reporting system comprising:
an ultrasound transducer probe;
a processor component;
the processor component configured to execute instructions to process ultrasound image information received from the ultrasound transducer probe into an ultrasound image and store the ultrasound image on a data storage component;
the processor component configured to receive examination information received from an input device, to store the examination information in the data storage component, and to automatically insert at least a portion of the examination information into a main ultrasound examination report and an additional examination report stored on the data storage component.

2. The system of claim 1, wherein the ultrasound imaging system and reporting system is a fetal ultrasound examination imaging system.

3. The system of claim 1, wherein the processor component comprises a first processor configured to execute the instructions to receive imaging information from the probe;
and a second processor configured to receive the examination information from the input device and to execute instructions automatically, without user interaction, to insert at least a portion of the examination information into the main ultrasound examination report and the additional examination report.

4. The system of claim 3, wherein the data storage component is coupled to the second processor or the first processor.

5. The system of claim 1, wherein the processor component is configured to accept input to create a template-based examination report, wherein the template-based examination report is stored in the data storage component as the additional examination report or is stored in the data storage component as the main examination report.

6. The system of claim 1, wherein the examination information comprises patient demographic information, patient measurement information obtained by measuring the ultrasound image, and practitioner diagnostic notes.

7. The system of claim 6, wherein the patient demographic information and patient measurement information obtained by measuring the ultrasound image includes fetal examination information and fetal measurement information.

8. The system of claim 7, wherein the fetal examination information and fetal measurement information includes information for multiple fetuses.

9. The system of claim 1, wherein the additional examination reports are selected from one or more of a group consisting of: a perinatal consult report, a report that includes data relevant for a patient's general practitioner or specialist, a diabetic patient log report, a telephone call memo, and a medication list.

10. The system of claim 1, wherein the processor component is configured to receive input that finalizes the main ultrasound examination report and all additional examination reports simultaneously, wherein upon finalization further changes to the main examination report and all additional examination reports are prohibited without receiving additional authorizing input.

11. A method for operating an ultrasound imaging and reporting system comprising:
receiving an ultrasound image from an ultrasound machine and storing the ultrasound image on a data storage component;
receiving examination information from an input device component and storing the examination information on the data storage component; and
automatically inserting at least a portion of the examination information into the main examination report and an additional examination report.

12. The method of claim 11 further comprising performing an ultrasound examination with an ultrasound transducer probe to obtain ultrasound image information;
processing the ultrasound image information to form an ultrasound image;
wherein the ultrasound examination is performed on a fetus.

13. The method of claim 11, further comprising automatically and without user interaction inserting the ultrasound image or a modified version thereof into the main examination report and the additional examination report.

14. The method of claim 11, wherein the examination information comprises patient demographic information, information regarding the examination, patient measurement information obtained by measuring the ultrasound image, and practitioner diagnostic notes.

15. The method of claim 14, wherein upon receiving the examination information from the input device component, displaying the examination information on a graphical user interface in fields of the main examination report.

16. The method of claim 15, further comprising:
receiving a request to display the additional examination report on the graphical user interface and in response retrieving from the data storage component and displaying the additional examination report with at least a portion of the examination information already entered into fields of the additional examination report.

17. The method of claim 16, wherein upon receiving the request to display the additional examination report, a processor component executes instructions to parse the examination information in data storage associated with the main examination report, for shared information that is associated with the fields in the additional examination report and automatically display the additional examination report with shared information already entered into the fields of the additional examination report.

18. The method of claim 11, wherein the additional report is selected from one or more of the group consisting of: a perinatal consult report, a report that includes data relevant for a patient's general practitioner or specialist, a diabetic patient log report, a telephone call memo, and a medication list.

19. The method of claim 11, further comprising receiving input to finalize the main examination report, and in response finalizing the main examination report and all additional examination reports simultaneously, wherein upon finalizing further changes to the main examination report and all additional examination reports are prohibited without receiving additional authorizing input.

20. An ultrasound imaging reporting system comprising:
a processor component;
the processor component configured to execute instructions to receive an ultrasound image from an ultrasound machine and store the ultrasound image on a data storage component;
the processor component configured to receive examination information received from an input device and store the examination information in the data storage component;
the processor component configured to execute instructions to automatically insert at least a portion of the examination information into a main examination report and an additional examination report stored on the data storage component.
